(19) United States
(12) Patent Application Publication
(10) Pub. No.: US 2025/0040916 A1
(43) Pub. Date: Feb. 6, 2025

(54) MEDICAL BRUSHES WITH MULTIPLE DEGREES OF FREEDOM AND RELATED METHODS

(71) Applicants: Boston Scientific Medical Device Limited, Galway (IE); Boston Scientific Scimed, Inc., Maple Grove, MN (US)

(72) Inventors: Deepak Kumar SHARMA, Muzaffarnagar (IN); Sharath Kumar G, Kanakapura (IN); Paul SMITH, Smithfield, RI (US); Scott E. BRECHBIEL, Burlington, MA (US)

(73) Assignees: Boston Scientific Medical Device Limited, Galway (IE); Boston Scientific Scimed, Inc., Maple Grove, MN (US)

(21) Appl. No.: 18/787,258

(22) Filed: Jul. 29, 2024

Related U.S. Application Data
(60) Provisional application No. 63/517,001, filed on Aug. 1, 2023.

(51) Int. Cl. A61B 10/02

(52) U.S. Cl. CPC A61B 10/02; A61B 2010/0208; A61B 2010/0216

(57) ABSTRACT

A cytology brush includes a shaft including a fluid lumen configured for passage of a fluid; a brush body including: a fixed element; and a moveable element with a radial outward surface and a plurality of bristles, wherein the bristles protrude externally from the radial outward surface. The moveable element is configured to receive the fluid through the fluid lumen to expand from an unexpanded configuration to an expanded configuration and in the unexpanded configuration of the moveable element, the bristles are in a retracted configuration and in the expanded configuration the bristles are in a deployed configuration.

### CROSS-REFERENCE TO RELATED APPLICATIONS

This application claims the benefit of priority to U.S. Provisional Application No. 63/517,001, filed on Aug. 1, 2023, which is incorporated by reference herein in its entirety.

### TECHNICAL FIELD

Aspects of this disclosure generally relate to medical brushes with multiple degrees of freedom and related methods. In particular, aspects of this disclosure relate to cytology brushes having brushes with multiple degrees of freedom and methods related to using such cytology brushes.

### BACKGROUND

Analyzing cell samples can be a helpful tool in the diagnosis and treatment of disease. Undiagnosed pain, unusual symptoms, unusual or abnormal samples, or other factors can require further investigation, and such investigation often necessitates using advanced medical devices. Medical devices, such as cytology brushes or other suitable devices, may be employed for a variety of diagnostic and surgical procedures. Many of these procedures involve delivering the cytology brush to an internal stricture (e.g., a biliary stricture) using a wire guided catheter. Diagnosis procedures often involve the removal of a portion of suspected cancerous tissue by collecting a sample of a mucosal tissue layer off the surface of a lumen within the biliary track. Diagnosing biliary strictures, done typically with cytology brushes, can be difficult because of a low cancer sensitivity rate, which can produce a false-negative diagnosis during an endoscopic retrograde cholangiopancreatography (ERCP) procedure. Low sensitivity is frequently linked to inadequate tissue sampling, which can be a limiting factor in the detection of potential malignancy.

Diagnosis procedures can involve removal of suspected cancer by collection of a sampling of a mucosal tissue layer off the surface of a lumen within the biliary track. Although current brushes may have a degree of freedom of movement (i.e., distally in and proximally out of a patient's body, as guided along a guidewire), the brush may not be capable of entering or crossing a stricture. For example, a tip or distal end of a brush includes radially or outwardly extending bristles, and the bristles may redirect or otherwise impede the delivery or movement of brush (e.g., when entering a stricture). In some cases, for example, brush movement may terminate in a dilated region or pocket in front of a stricture. Brushes with additional degrees of freedom of movement, for example, with an ability to expand/collapse, may provide additional functionality with respect to passing a stricture or otherwise collecting a sample.

The devices and methods of this disclosure may rectify one or more of the deficiencies described above or address other aspects of the art. It is to be noted, however, that the scope of subject matter of this application is defined by the features listed in the claims, and not an ability to rectify any particular deficiency.

### SUMMARY

Each of the aspects disclosed herein may include one or more of the features described in connection with any of the other disclosed aspects.

Aspects of the disclosure relate to, among other things, systems, devices, and methods for recovering a sample from a subject. Aspects of this relate to, among other things, medical brushes with multiple degrees of freedom and related methods.

In some aspects, a cytology brush may include a shaft including a fluid lumen configured for passage of a fluid, and a brush body. The brush body may include a fixed element, and a moveable element with a radial outward surface and a plurality of bristles. The bristles may protrude externally from the radial outward surface. The moveable element can be configured to receive the fluid through the fluid lumen to expand from an unexpanded configuration to an expanded configuration. In the unexpanded configuration of the moveable element, the bristles may be in a retracted configuration, and, in the expanded configuration, the bristles may be in a deployed configuration.

The cytology brush may include one or more of the following aspects. The moveable element may be a balloon that is divided into a plurality of balloon sections. Each balloon section may include one or more bristles on an external surface of the balloon section. The moveable element may include three balloon sections: a distal balloon section, a middle balloon section, and a proximal balloon section. The bristles on the middle balloon section may have a lowest stiffness. The bristles in the middle balloon section may extend the greatest distance from a central axis of the cytology brush. The balloon may be divided into three sections: a proximal balloon section, a middle balloon section, and a distal balloon section, each section comprising a distal sidewall and a proximal sidewall. An outer wall of one or more of the balloon sections may have a different hardness than an outer wall of other balloon sections.

The one or more balloon sections with a lowest hardness rating may expand furthest from a central axis of the shaft. Each of the balloon sections may include a distal sidewall and a proximal sidewall. When the balloon section is in the unexpanded configuration, a subset of bristles extend proximally from the distal sidewall and another subset of bristles extend distally from the proximal sidewall. The fluid may be air. The air may be delivered by a syringe operatively connected to the fluid lumen. The cytology brush may include four fluid lumen equally spaced about a circumference of the shaft to deliver fluid to respective portions of the brush. When the bristles are in the deployed configuration, a central axis of each of the bristles may be perpendicular to a central axis of the shaft. The balloon may be biased toward a collapsed configuration. The fixed element may include a distal end wall and a proximal end wall. The distal end wall and the proximal end wall may be spaced apart longitudinally and connected by a plurality of longitudinal sidewalls extending between the distal end wall and the proximal end wall. The balloon may form an air tight seal between each of the distal sidewalls, the proximal sidewall, and the longitudinal sidewalls forming one or more chambers for receiving fluid to expand and collapse the balloon between the expanded configuration and the unexpanded configuration such that the bristles move between the deployed configuration and a retracted configuration. The bristles may form four columns of pairs of bristles in a longitudinal direction of the brush and overlap in the undeployed configuration. The balloon may be caused to move between the expanded configuration and the unexpanded configuration by fluid pressure.

In another aspect, a cytology brush may include a shaft and a brush body. The shaft may include a guidewire lumen configured for passage of a guidewire and a plunger moveable to cause the brush to move between the various configurations. The brush body may include a fixed element and a balloon. The balloon may include an outer surface including bristles protruding externally from the outer surface of the balloon. The balloon may be configured to receive at least a portion of the plunger to expand from an unexpanded configuration to an expanded configuration. In the unexpanded configuration of the balloon, the bristles may be in an undeployed configuration, and, in the expanded configuration, the bristles may be in a deployed configuration.

The cytology brush may include one or more of the following aspects. The cytology brush may include a wire connected between the plunger and a distal end of the brush body, extending in a longitudinal direction over the balloon. The wire may be biased toward an expanded configuration. The plunger may be movable to flex the wire inward, causing the balloon to move from the expanded configuration to the unexpanded configuration. The balloon may be nylon, silicone, or polymer based.

In yet another example, a method of collecting a tissue sample may include navigating a guidewire relative to tissue at treatment site, advancing a cytology brush over guidewire to the treatment site, and transitioning the cytology brush from an unexpanded configuration to an expanded configuration. In transitioning the cytology brush from the unexpanded configuration to the expanded configuration, bristles on one or more sections of the cytology brush may move from an undeployed configuration to a deployed configuration. The undeployed configuration may include corresponding pairs of bristles on the section at least partially overlapping, and the deployed configuration may include corresponding pairs of bristles on the section not overlapping. The method further may include collecting tissue on the bristles by moving the cytology brush in one or more of: longitudinally along the guidewire, circumferentially around the guidewire, and between the unexpanded and expanded configurations, transitioning the cytology brush from the expanded configuration to the unexpanded configuration, and removing the cytology brush from the body lumen of the patient and collecting the tissue collected in the bristles.

The method may also include one or more of the following aspects. The cytology brush may include one or more balloons. The method may further include causing at least one of the one or more balloons to expand to the expanded configuration thereby causing the bristles to move to the deployed configuration.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 illustrates a distal portion of an exemplary medical device, according to aspects of this disclosure.

FIG. 2A illustrates another view of the exemplary medical device of FIG. 1.

FIG. 2B illustrates a partially transparent view of a proximal portion of the exemplary medical device of FIG. 1.

FIG. 3 illustrates particular aspects of the distal portion of the exemplary medical device of FIG. 1.

FIG. 4 illustrates additional features of the exemplary medical device of FIG. 1.

FIG. 5A illustrates a distal portion of another exemplary medical device.

FIG. 5B illustrates a distal portion of the exemplary medical device of FIG. 5A.

FIG. 5C illustrates another distal portion of the exemplary medical device of FIG. 5A.

FIG. 6A illustrates a distal portion of yet another exemplary medical device.

FIG. 6B illustrates another view of the exemplary embodiment of the medical device of FIG. 6A.

FIG. 7A illustrates a distal portion of another exemplary medical device.

FIG. 7B illustrates another view of the exemplary embodiment of the medical device of FIG. 7A.

FIG. 8 illustrates a related method of using an exemplary medical device, such as the medical device of FIG. 1.

### DETAILED DESCRIPTION

Reference will now be made in detail to examples of this disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body of a patient or other subject or closer to a user, such as a medical professional, holding or otherwise using the medical device. In contrast, “distal” refers to a position relatively further away from the medical professional or other user holding or otherwise using the medical device, or closer to the interior of the patient's body. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

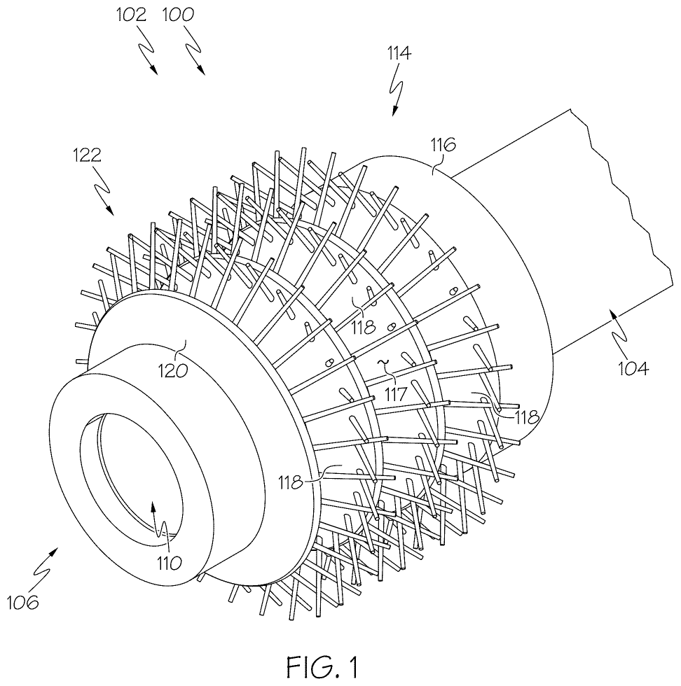

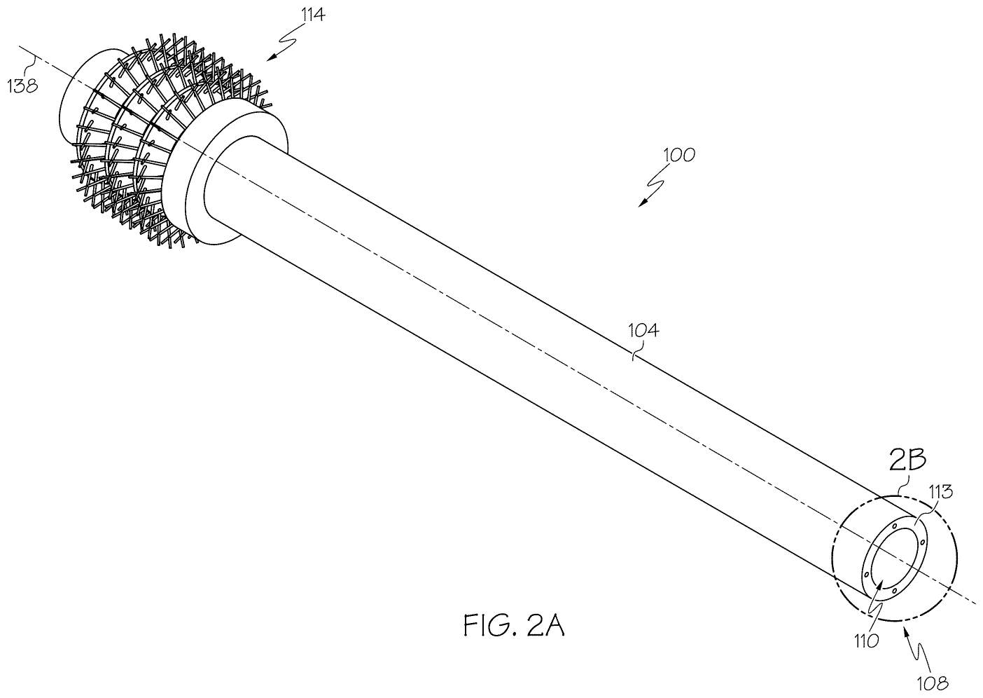

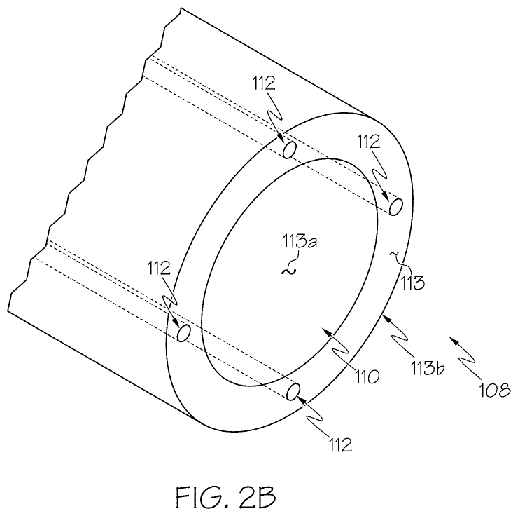

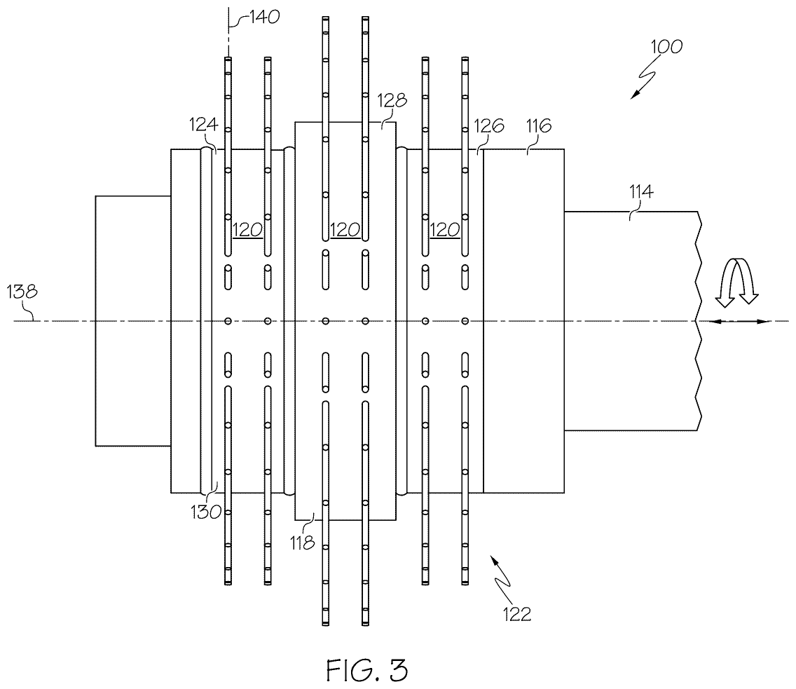

FIGS. 1-4 depict a medical device 100 that may be configured to fit over a portion of a guidewire (not shown). The medical device 100 may include a cytology brush 102 (or simply “brush”) and a shaft 104 that extends between a first or distal end 106 and a second or proximal end 108 (FIG. 2A) along a central longitudinal axis 138 (FIG. 2A, FIG. 3) of the medical device 100. In some aspects, one or more portions of the shaft 104 are at least partially flexible, and the brush 102 may include a plurality of bristles 122. The brush 102 may be operable in multiple degrees of freedom. For example, the brush 102 may generally be configured to be delivered and deployed to a treatment site, collect a sample of tissue at the treatment site by motion in one or more of the multiple degrees of freedom, and to be retracted or removed from the body of a patient. In these aspects, the sample collected at the treatment site may be collected and analyzed by a medical professional(s) to produce a test result (e.g., biopsy, etc.). Moreover, as discussed in detail below, the brush 102 may be manipulated to transition from an undeployed or retracted configuration to a deployed configuration for the sample collection. Then, the brush 102 may be manipulated to transition from the deployed configuration to the retracted configuration, for example, to help retain and/or protect the sample, for removal of the brush 102 and the sample from the body.

In some aspects, the shaft 104 includes a guidewire lumen 110 for receiving a guidewire (not shown), such that the brush 102 can move along (e.g., slide along) the guidewire to the treatment site in the patient's body. Hence, the guidewire may be placed inside the patient at or near the treatment site before insertion and placement of the brush 102. In some embodiments, the shaft 104 also includes one or more fluid lumens 112 (FIG. 2B).

The fluid lumens 112 may extend longitudinally through respective portions of shaft 104. For example, shaft 104 may include a thickness 113, for example, radial thickness if shaft 104 includes a circular lateral cross-section. Thickness 113 may be formed by an inner wall 113a and an outer wall 113b, with fluid lumens 112 extending through shaft 104 between inner wall 113a and outer wall 113b. The fluid lumens 112 may allow the flow or passage of a fluid (e.g., water, saline, air, etc.) that may be used to operate the brush 102, as explained in greater detail below. As shown in, for example, FIGS. 2A and 2B, shaft 104 may include four fluid lumens 112, and the four fluid lumens 112 may be equally spaced about the shaft 104 (i.e., each at an equal radial distance from a center of the shaft 104 and with an equivalent angular spacing around the center of the shaft 104). Nevertheless, it is noted that this disclosure is not so limited. For example, shaft 104 may include one, two, three, five, or more fluid lumens 112, and/or the fluid lumens 112 may be unevenly spaced about the internal portion of shaft 104.

Referring to FIGS. 1, 3, and 4, the brush 102 generally comprises a fixed structure 116 and a flexible structure 118. The flexible structure 118 may be a moveable element that may have an expandable outward facing surface that may expand and contract (similar to the expansion and contraction of, for example, a balloon) between an unexpanded configuration and an expanded configuration. The flexible structure 118 transitioning between the unexpanded configuration and the expanded configuration may cause the bristles 122 of the brush 102 to be moved from a relaxed configuration (or “undeployed configuration”) to a deployed configuration and vice versa based on the expansion. The bristles 122 may protrude externally from a radial outward surface 117 of the flexible structure 118. When in the bristles 122 are in the deployed configuration, the bristles 122 may extend generally perpendicular to the central longitudinal axis 138 of the shaft 104. In some embodiments, the bristles 122 may form pairs of bristles 122, for example, that extend parallel to one another in the deployed configuration and that at least partially overlap when the bristles 122 are in the undeployed configuration.

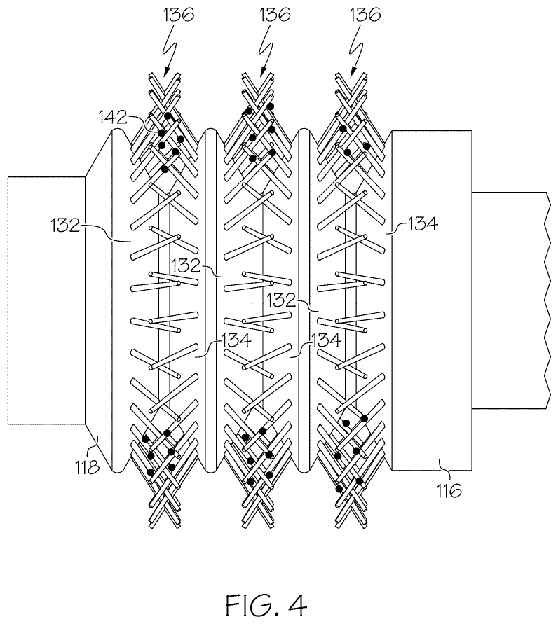

In some embodiments, the flexible structure 118 may be biased toward a collapsed configuration. Additionally or alternative, the flexible structure 118 may be divided into one or more sections (e.g., as shown in FIG. 3, three sections: a distal section 124; a middle or intermediate section 128; and a proximal section 126). As in FIG. 4, each section 124, 126, 128 may generally comprise a distal sidewall 132 and a proximal sidewall 134. The bristles 122 may project from distally or proximally from either or both of the distal sidewall 132 and the proximal sidewall 134. Subsets of bristles 122 from adjacent walls or portions may at least partially cross or overlap one another or otherwise interact to form a trap 136 that may help to trap or otherwise retain tissue 114 (FIG. 4). In these aspects, the arrangement of the bristles may help to increase the amount of tissue 114 collected and retained with the brush 102.

One or more of sections 124, 126, 128 of the brush 102 may have different characteristics. For example, the sections 124, 126, 128 may have different material properties that allow the different sections to operate differently from one another, which may help to collect and/or retrain sample(s), as explained in greater detail below. For example, the different material properties and/or the flow of fluid to brush 102 may cause at least one of the sections to expand and/or contract differently than the other sections.

FIGS. 3 and 4 show the brush 102 in an expanded configuration and a deflated configuration, respectively. In the particular exemplary embodiment depicted in FIG. 3, the material used to form the ballooning portion of the middle section 128 can exhibit a lower hardness and/or greater flexibility than that exhibited by the material(s) used to form the ballooning portions of the distal section 124 and the proximal section 126. In some embodiments, the material may be nylon, silicone, or polymer based. All of the sections may be formed from the same material or may formed from different materials. In some embodiments, sections that expand with a greater flexibility may be formed with a thinner wall and/or more flexible material, and sections that expand with a lesser flexibility may be formed with a thicker wall and/or a less flexible material. In the exemplary embodiment, the middle section 128 has the lowest hardness rating. Accordingly, as shown in FIG. 3, the outer wall of the middle section 128 may extend furthest from the central longitudinal axis 138. In some embodiments, the bristles 122 of the middle section 128 may extend further from the central longitudinal axis 138 of the brush 102 than the bristles 122 on one or more of the distal section 124 or the proximal section 126. In these aspects, the bristles 122 of the middle section 128 may collect sample or portions of the patient's anatomy that are further from the central longitudinal axis 138 of the brush 102. In some embodiments, the bristles 122 and/or the various sections may have substantially equivalent properties, giving the brush 102 a constant diameter. Further, the smaller size of the distal section 124 and the proximal section 126 may help to allow for the brush 102 to be gradually introduced and/or removed from a smaller anatomy (e.g., a stricture).

Additionally, the bristles 122 of different sections or of the same section may have different characteristics. In some aspects, the bristles 122 on the brush 102 are all of similar length (as shown). However, this disclosure is not so limited. In some aspects, the bristles 122 may include different lengths, cross-sectional sizes, spacings, etc. The bristles 122 could also have different stiffness (e.g., as measured using a durometer), durability, or other material characteristics. In some embodiments, the bristles 122 are arranged in different formations than those shown in FIGS. 1-4. For example, a brush section could have two or more rows of bristles on a single wall (e.g., a short row of bristles and a tall row of bristles), which may help to increase the amount or volume of sample collected.

Still referring to the embodiment generally depicted in FIGS. 1-4, the brush 102 can be transitioned between the expanded and contracted configurations via the delivery and/or removal of a working fluid. For example, a user may force a fluid (e.g., water, saline, air, etc.) through the fluid lumens 112 using a syringe or other device operatively connected to the shaft 104 and configured to impart a positive relative pressure to the fluid to force the fluid through the fluid lumens 112 and into the brush 102. Entrance of the fluid may cause the expandable portions of the brush 102 to expand, thus transitioning the bristles 122 to the deployed configuration. Applying a negative relative pressure to the working fluid (e.g., air, water, saline, etc.) may reduce the pressure within the brush 102 and allow the expandable portions to relax. In some embodiments, for example embodiments having a different number of fluid lumens 112 as that shown in FIGS. 2A and 2B, the working fluid may be delivered distally through the fluid lumens 112 and then enter a common chamber or fluid distribution ring inside a distal portion of the fixed structure 116 where the fluid lumens 112 may be fluidly coupled before entering the balloon or other operable section such that it enters various portions of the expandable brush with a relatively equivalent pressure between various portions.

Additionally, the brush 102 may be rotatable around the central longitudinal axis 138 (FIGS. 2A and 3) to collect the tissue sample. The brush 102 may be rotatable by a user via one or more tools or devices external to the patient's body (e.g., at a handle at a distal end of the medical device 100). The user may rotate the brush 102 about the central longitudinal axis 138 such that the bristles come into increasing contact with patient tissue to increase the amount of sample collected with the bristles 122. Additionally, the brush 102 may advance distally and retract proximally using, for example, a pushing and pulling force to move the brush 102 through the patient to a treatment site (e.g., along a guidewire). Additionally, the brush may expand and contract as explained hereinabove. Hence, the brush 102 may be manipulated through at least six degrees of freedom to collect tissue/sample.

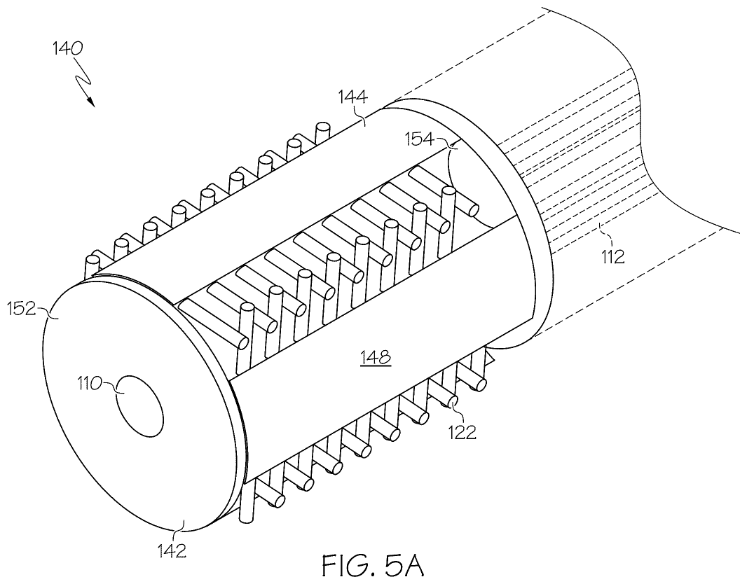

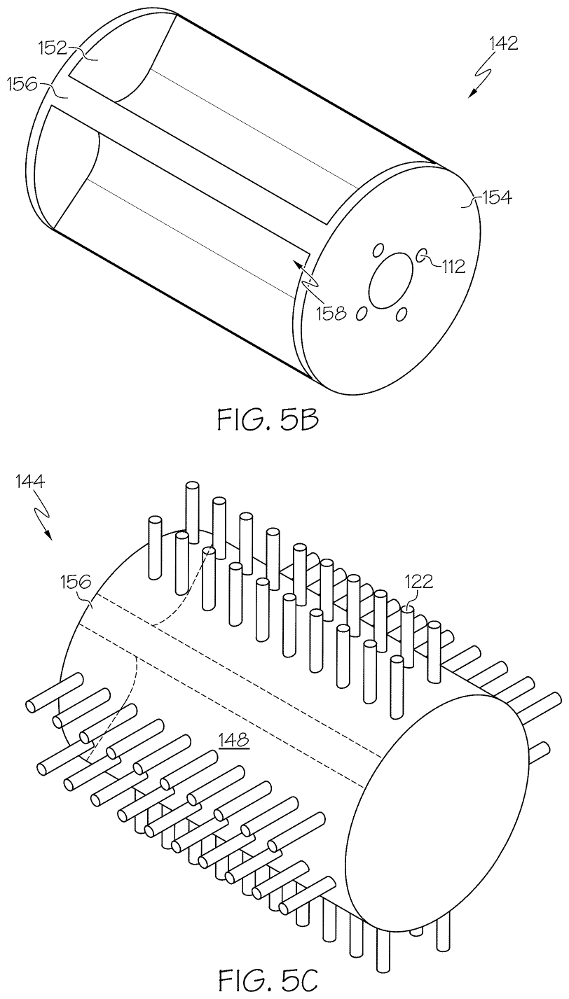

Referring to FIGS. 5A-5C another embodiment of a medical device for collecting a tissue sample is shown. FIG. 5A shows a cytology brush 140 (or simply “brush”) that includes a fixed structure 142 and a flexible structure 144 with bristles 122 on an external surface 148 of the flexible structure 144. As shown best in FIG. 50, the flexible structure 144 may be a balloon-like structure or flexible sleeve that is positioned over the fixed structure 142 shown in FIG. 5B. The fixed structure 142 may include longitudinal sidewalls 156, a distal end wall 152, and a proximal end wall 154. A longitudinal sidewall 156 is shown in relief in FIG. 5C. The longitudinal sidewalls 156, distal end wall 152, and proximal end wall 154 may form chambers 158. The chambers 158 may be separated radially, and the various walls and flexible structure 144 may help to form an air tight seal around each of the chambers 158. As discussed above, one or more fluids may be delivered to and removed from the chambers 158 through fluid lumens 112 (FIG. 5B), for example, to transition the flexible structure 144, and hence the bristles 122, from a relaxed configuration to an expanded configuration.

With the flexible structure 144 in place generally surrounding the fixed structure 142, fluid may be provided to and removed from the chambers 158 to expand and contract the flexible structure to deploy and relax the bristles 122 thereon. When the bristles 122 transition from the deployed configuration to the relaxed configuration, two or more of the bristles 122 may at least partially overlap to help trap and/or retain tissue. The trapped and/or retained tissue may be removed from the patient and sampled, as otherwise mentioned herein. In some embodiments, there may be more than one type of flexible structure 144 that is adaptable to fit over the fixed structure 142 (e.g., adaptable sleeves). Providing various types of structures can enable varying operational characteristics of the brush 140 based on the operational characteristics of the particular flexible structure chosen (e.g., more or fewer bristles, greater or less hardness, etc.)

As shown in FIG. 5C, the bristles 122 may be arranged in parallel rows (e.g., two parallel rows) and may be generally of equivalent length. However, this is not required and any arrangement of bristles 122 on the external surface 148 is possible.

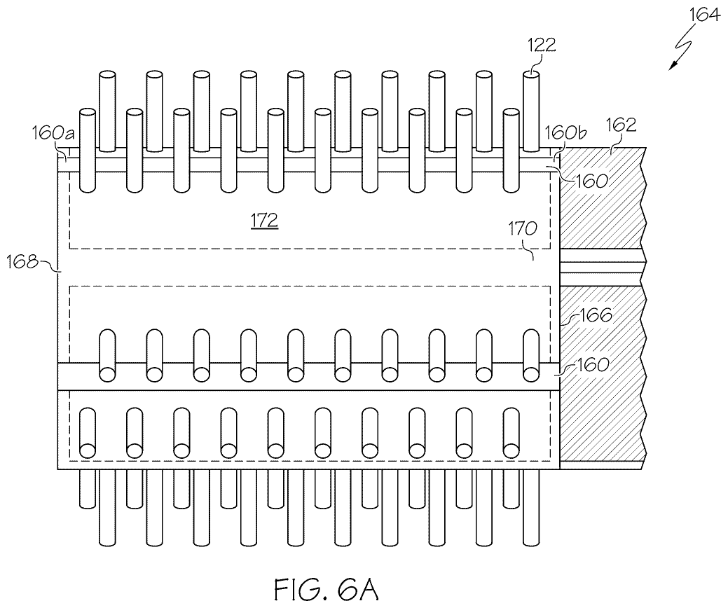

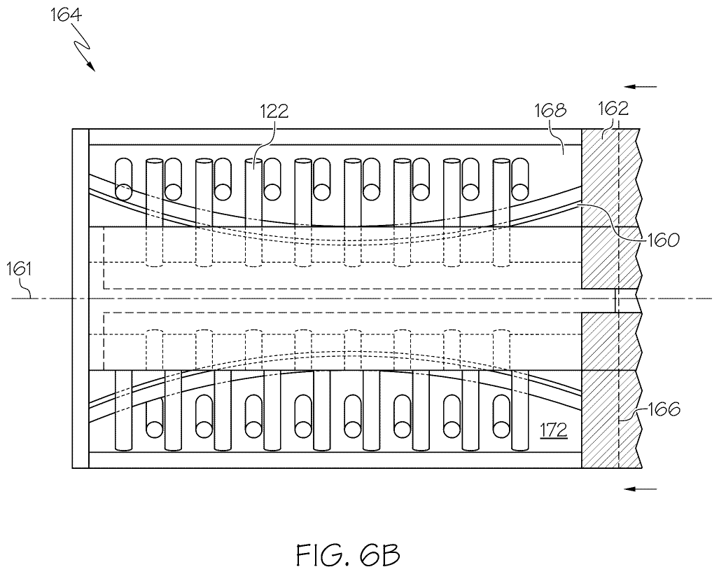

FIGS. 6A and 6B show an embodiment of a cytology brush 164 (or simply “brush”) having a flexible element 168 and a fixed element 170 similar to the flexible structure 144 and the fixed structure 142 of FIGS. 5A-5C. That is, the flexible element 168 may be a flexible sleeve that is positioned over the fixed element 170 to cover one or more chambers (not shown) on the fixed element 170 such that portions of the flexible element 168 can fold into the chambers.

The brush 164 further includes a plunger 162 and a spring 160 (which may be formed by, for example, a flat wire). As shown in FIG. 6A, the plunger 162 is in a first position and the spring 160 is in a relaxed position. In FIG. 6B, the plunger 162 is urged distally toward a second position to be inserted into a chamber 166 of the fixed element 170. Insertion of the plunger 162 causes the spring 160 to flex or bend inward (e.g., such that a middle portion of the wire 160 flexes radially inward to a longitudinal axis 161). Because the distal end 160a and the proximal end 160b of the spring 160 are fixed to the fixed element 170 over an external surface 172 of the flexible element 168, the spring 160 bends inward when compressed along its longitudinal direction by the plunger 162. The spring 160 forces the flexible element 168 inward as the spring 160 flexes inward. When the flexible element 168 is moved inward, at least a subset of the bristles 122 on the external surface 172 of the flexible element 168 move into the retracted configuration, at least partially overlapping and helping to trap or retain one or more tissue samples while the brush 164 is removed from the patient's body.

In some embodiments, the spring 160 may be coupled to the flexible element 168. When the plunger 162 is moved proximally allowing more space for the spring 160 to move to its first position, the spring 160 may pull the flexible element 168 back to the expanded configuration, causing the bristles 122 to extend (e.g., to collect a sample). In other embodiments, the flexible element 168 may be biased toward the expanded configuration, and the flexible element 168 may or may not be coupled to the spring 160 such that the flexible element 168 defaults to the expanded configuration whenever the spring 160 is not moving the flexible element 168 into the retracted configuration.

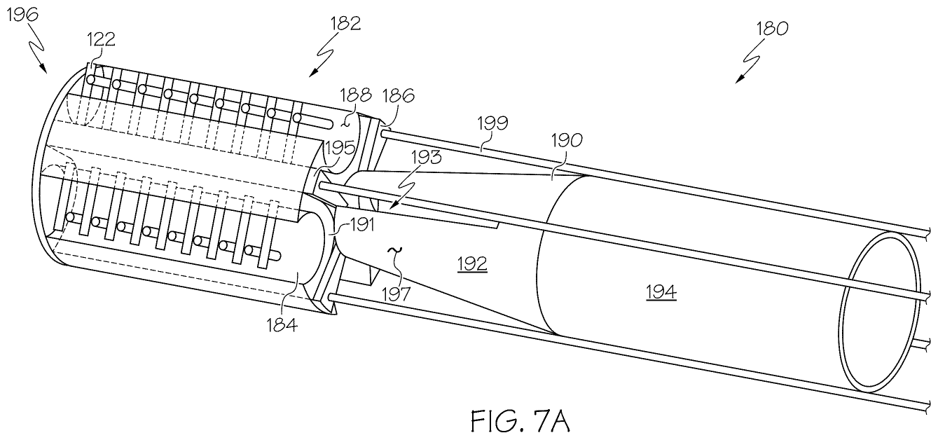

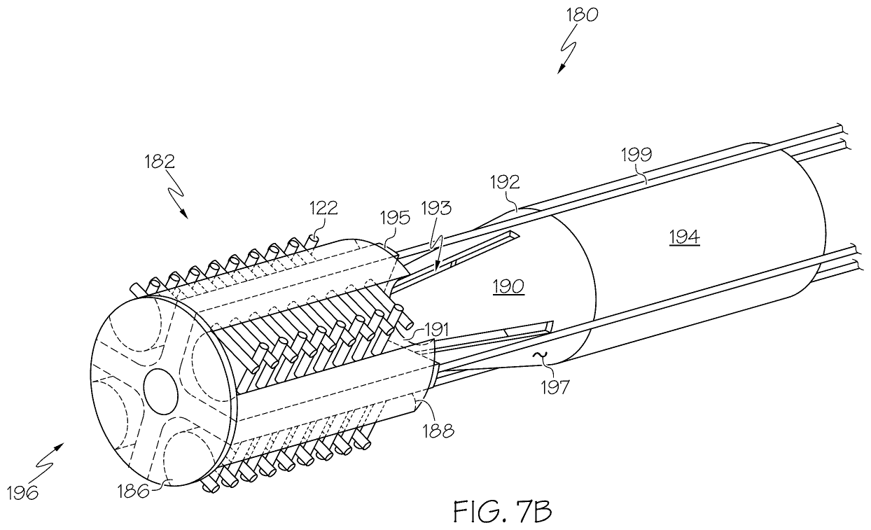

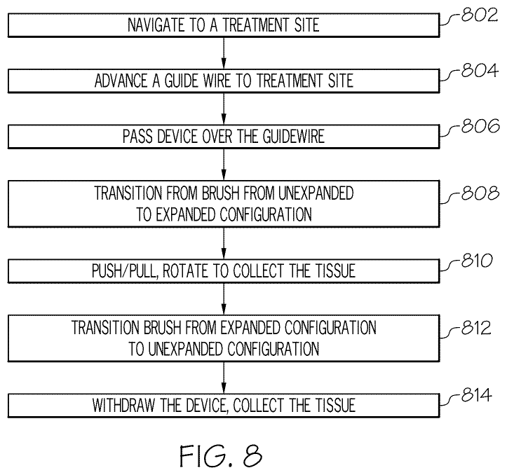

FIGS. 7A and 7B show another embodiment of a medical device 180 including a cytology brush 182 (or simply “brush”). The brush 182 includes an expandable element 184 and a fixed element 186. Wires 199 connect to the fixed element 186. The wires 199 may connect to the fixed element 186 at longitudinal sidewalls 195. The expandable element 184 includes bristles 122 on its outer surface. The expandable element 188 may expand as the expandable element 188 is pushed or otherwise urged outward by distal movement of a plunger 190. The plunger 190 may be configured to fit between the expandable element 184 and the fixed element 186 when the plunger 190 is extended distally. When the plunger 190 is positioned between the expandable element 184 and the fixed element 186, the plunger 190 may at least partially force the expandable element 184 radially outward, for example, to transition the expandable element 184 and its bristles from a retracted configuration (FIG. 7A) to an expanded configuration (FIG. 7B). It is noted that the fixed element 186 may be movable (e.g., longitudinally), but does not expand or otherwise change in size or shape during the operation of medical device 180.

In some embodiments, the plunger 190 can have a distal section 192 and a proximal section 194. The distal section 192 can have a tapered or partially tapered shape (e.g., a partially or truncated conical shape). The proximal section 194 can have a constant cross sectional shape along its length. The distal section 192 can have cut away portions 193, which may surround longitudinal walls 195 of the fixed element 186, for example, when the plunger 190 is advanced distally and/or when the fixed element 186 is retracted proximally. The cut way portions 193 may separate projections 197 of the distal section 192.

In embodiments, one or more of the plunger 190 and the brush 182 may be movable relative to the other of the plunger 190 and the brush 182. The relative movement may cause the distal section 192 of the plunger to be positioned radially inward or behind the expandable element 184 with respect to an outer surface of the brush 182. For example, in one aspect, the plunger 190 may be distally advanced relative to the brush 182 such that at least a portion of the distal section 192 is positioned radially inward or behind at least a portion of the expandable element 184. In another aspect, the brush 182 may be proximally retracted relative to the plunger 190. In this aspect, one or more of the wires 190 may be proximally retracted to proximally retract the brush 182 relative to the plunger 190. Based on the relative longitudinal motion, the expandable element 184 can expand outward, causing the bristles 122 to expand outward, for example, for sample collection. In some embodiments, the distal section 192 may extend forward (i.e., distal) of a distal end 196 of the fixed element 186 until the proximal section 194 is behind (e.g., radially inward of) all or a portion of the expandable element 188, such that the bristles 122 nearest the distal end 196 of the brush 182 are as extended as the bristles at an opposite end.

FIG. 8 depicts an exemplary method of using a medical device, such as the medical device 100 described above. While reference is made to the exemplary embodiment shown in FIGS. 1-4 with respect to the method of FIG. 8, it is understood that the principles of operation described here are applicable to the other embodiments shown and described herein and to other embodiments meeting the features of the claims listed below. A sheath or insertion device (not shown) may be inserted into the patient's body through an incision or natural orifice, and may be navigated by a user (e.g., a physician) to a treatment site (e.g., a stricture) at step 802. For example, an endoscope or other medical device may be inserted into the patient's body and navigated using images or other navigation tools through a lumen in the patient's body until a distal end reaches the target area, which may be a treatment site (e.g., a treatment site for obtaining a tissue sample).

A guidewire may be inserted into a lumen of the sheath or the insertion device, and the guidewire may be advanced to the treatment site of the patient's body at step 804. The guidewire may be advanced to the treatment site within the lumen of the sheath or the insertion device, for example. A pushing force on a proximal portion of the guidewire may be used to direct the guidewire to the treatment site. In some aspects, the sheath or the insertion device may remain in the patient near the treatment site or along some portion of the path used to reach the treatment site. In other aspects, the sheath or the insertion device may be removed from the patient once the guidewire is in place near the treatment site.

The brush 102 may be slideably placed or positioned on the guidewire from outside the patient's body (e.g., by positioning the proximal end of the guidewire within the guidewire lumen 110), and the brush 102 may be passed over a length of the guidewire to the treatment site at step 806. Because the brush 102 is configured such that the guidewire fits within the guidewire lumen 110, the brush 102 may be passed along the guidewire until the brush 102 is at the treatment site.

Once the brush 102 is at the treatment site, the brush 102 may be transitioned from an unexpanded configuration to an expanded configuration. In some embodiments, for example, this may cause a balloon portion to expand thereby causing the bristles to push against the tissue to allow the bristles to disrupt the tissue at step 808. The balloon portion may be filled with a working fluid such as air, for example. In some embodiments, a user may use a syringe or other pump to force the working fluid into the balloon portion through the fluid lumens 112. In some embodiments, the resting position of the balloon portion may be in an expanded configuration, and a user may apply a suction pressure (negative relative pressure) to keep the brush 102 in a contracted configuration during delivery of the brush, relieving the negative relative pressure once the brush 102 is at the treatment site to allow the bristles 122 of the brush 102 to expand to contact tissue at the treatment site. That is, expansion of the bristles 122 may push the bristles 122 against the tissue, allowing the bristles 122 to disrupt the tissue.

At the treatment site, with the brush 102 in an expanded configuration, the user may push, pull, and/or rotate (i.e., circumferentially around the guidewire) the brush 102 to collect tissue at step 810. In embodiments, a distal end of the brush 102 may be manipulable by the user to move the brush 102. For example, the brush 102 may be pushed, pulled, and/or rotated using a device such as a wire or other device connected to the shaft 104. In some embodiments, the shaft 104 may stretch from the treatment site all the way to the user of the medical device 100.

At step 812, the user may cause the brush 102 to transition from the expanded configuration to the unexpanded configuration. For example, in embodiments having a balloon feature, a user may allow the balloon to collapse, entering the relaxed state or contracted state. In some embodiments, the contracted state may require providing a negative pressure to an expandable portion. For example, in embodiments in which a balloon is biased in an expanded position. With the balloon in the contracted state, a user may withdraw the balloon from the patient, to remove the tissue sample from the patient at step 814. In other embodiments, a user may remove a plunger or other implement to allow an expandable section to contract. In embodiments, the brush 102 may be removed from the patient in a proximal direction as compared with the guidewire, which may remain in the patient until a sufficient amount of tissue sample has been verified to have been collected. In other embodiments, the guidewire and the brush 102 may be removed simultaneously or together.

Embodiments of the present disclosure may be applicable to various and different medical or non-medical procedures. In addition, certain aspects of the aforementioned embodiments may be selectively used in collaboration, or removed, during practice, without departing from the scope of the disclosure.

While principles of this disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A cytology brush comprising:
a shaft including a fluid lumen configured for passage of a fluid;
a brush body comprising:
a fixed element; and
a moveable element with a radial outward surface and a plurality of bristles, wherein the bristles protrude externally from the radial outward surface;
wherein
the moveable element is configured to receive the fluid through the fluid lumen to expand from an unexpanded configuration to an expanded configuration and in the unexpanded configuration of the moveable element, the bristles are in a retracted configuration and in the expanded configuration the bristles are in a deployed configuration.

2. The cytology brush of claim 1, wherein the moveable element is a balloon that is divided into a plurality of balloon sections, each balloon section including one or more bristles on an external surface of the balloon section.

3. The cytology brush of claim 2, wherein the moveable element includes three balloon sections: a distal balloon section, a middle balloon section, and a proximal balloon section and the bristles on the middle balloon section have a lowest stiffness.

4. The cytology brush of claim 3, wherein the bristles in the middle balloon section extend the greatest distance from a central axis of the cytology brush.

5. The cytology brush of claim 2, wherein the balloon is divided into three sections: a proximal balloon section, a middle balloon section, and a distal balloon section, each section comprising a distal sidewall and a proximal sidewall.

6. The cytology brush of claim 5, wherein an outer wall of one or more of the balloon sections has a different hardness than an outer wall of other balloon sections.

7. The cytology brush of claim 6, wherein the one or more balloon sections with a lowest hardness rating expands furthest from a central axis of the shaft.

8. The cytology brush of claim 2, wherein each of the balloon sections includes a distal sidewall and a proximal sidewall and, when the balloon section is in the unexpanded configuration, a subset of bristles extend proximally from the distal sidewall and another subset of bristles extend distally from the proximal sidewall.

9. The cytology brush of claim 1, wherein the fluid is air, and wherein the air is delivered by a syringe operatively connected to the fluid lumen.

10. The cytology brush of claim 9, including four fluid lumen equally spaced about a circumference of the shaft to deliver fluid to respective portions of the brush.

11. The cytology brush of claim 1, wherein, when the bristles are in the deployed configuration, a central axis of each of the bristles is perpendicular to a central axis of the shaft.

12. The cytology brush of claim 2, wherein the balloon is biased toward a collapsed configuration.

13. The cytology brush of claim 2, the fixed element further comprising:
a distal end wall;
a proximal end wall, the distal end wall and the proximal end wall spaced apart longitudinally and connected by a plurality of longitudinal sidewalls extending between the distal end wall and the proximal end wall, wherein
the balloon forms an air tight seal between each of the distal sidewalls, the proximal sidewall, and the longitudinal sidewalls forming one or more chambers for receiving fluid to expand and collapse the balloon between the expanded configuration and the unexpanded configuration such that the bristles move between the deployed configuration and a retracted configuration.

14. The cytology brush of claim 13, wherein the bristles form four columns of pairs of bristles in a longitudinal direction of the brush and overlap in the undeployed configuration.

15. The cytology brush of claim 13, wherein the balloon is caused to move between the expanded configuration and the unexpanded configuration by fluid pressure.

16. A cytology brush comprising
a shaft including:
a guidewire lumen configured for passage of a guidewire; and
a plunger moveable to cause the brush to move between the various configurations;
a brush body comprising:
a fixed element; and
a balloon with an outer surface including bristles protruding externally from the outer surface of the balloon; wherein
the balloon is configured to receive at least a portion of the plunger to expand from an unexpanded configuration to an expanded configuration and in the unexpanded configuration of the balloon, the bristles are in an undeployed configuration and in the expanded configuration the bristles are in a deployed configuration.

17. The cytology brush of claim 16, further comprising:
a wire connected between the plunger and a distal end of the brush body, extending in a longitudinal direction over the balloon, wherein
the wire is biased in an expanded configuration and
the plunger is moveable to flex the wire inward, causing the balloon to move from the expanded configuration to the unexpanded configuration.

18. The cytology brush of claim 17, wherein the balloon is nylon, silicone, or polymer based.

19. A method of collecting a tissue sample, comprising:
navigating a guidewire relative to tissue at treatment site;
advancing a cytology brush over guidewire to the treatment site;
transitioning the cytology brush from an unexpanded configuration to an expanded configuration, wherein in an expanded configuration, bristles on one or more sections of the cytology brush move from an undeployed configuration to a deployed configuration, wherein the undeployed configuration includes corresponding pairs of bristles on the section at least partially overlapping, and wherein the deployed configuration includes corresponding pairs of bristles on the section not overlapping;
collecting tissue on the bristles by moving the cytology brush in one or more of: longitudinally along the guidewire, circumferentially around the guidewire, and between the unexpanded and expanded configurations;
transitioning the cytology brush from the expanded configuration to the unexpanded configuration; and
removing the cytology brush from the body lumen of the patient and collecting the tissue collected in the bristles.

20. The method of claim 19, wherein the cytology brush includes one or more balloons, the method further including causing at least one of the one or more balloons to expand to the expanded configuration thereby causing the bristles to move to the deployed configuration.
